(19) United States
(12) Patent Application Publication
(10) Pub. No.: US 2008/0188002 A1
(43) Pub. Date: Aug. 7, 2008

(54) METHOD AND APPARATUS FOR REMOVING SELENIUM OXIDE IN A SAMPLE, AND METHOD AND APPARATUS FOR MEASURING MERCURY IN COAL COMBUSTION EXHAUST GAS BY USING THE SAME

(76) Inventors: Junji Kato, Kyoto-shi (JP); Shigeyuki Akiyama, Otsu-shi (JP); Fujio Koga, Kyoto-shi (JP); Koji Ishikawa, Kyoto-shi (JP)

Correspondence Address: Snell & Wilmer L.L.P., Suite 1400, 600 Anton Boulevard, Costa Mesa, CA, 92626, US

(21) Appl. No.: 12/024,839

(22) Filed: Feb. 1, 2008

(30) Foreign Application Priority Data
Feb. 2, 2007 (JP) 2007-024456

(51) Int. Cl. G01N 31/12; G01N 33/20; B01D 3/00

(52) U.S. Cl. 436/81; 202/198; 422/80; 422/169; 95/227

(57) ABSTRACT

There is provided a method and apparatus for removing selenium oxide in a sample as well as a method and apparatus for measuring mercury in coal combustion exhaust gas by using the same. The apparatus for removing selenium oxide in a sample, comprising: (1) a heating introduction path for heating a sample, (2) a primary cooling unit having a flow path through which the heated sample flows countercurrently to cooling water, whereby the heated sample is mixed with, and cooled by, cooling water, (3) a secondary cooling unit having a spiral flow path for cooling the mixed gas and having a space for gas/liquid separation at the end of the spiral flow path, (4) a regenerator for introducing condensed water from the secondary cooling unit, and (5) a condensed water-cooling path for connecting the regenerator to the primary cooling unit.

### BACKGROUND OF THE INVENTION

1. Field of the Invention

The present invention relates to a method and apparatus for removing selenium oxide in a sample as well as a method and apparatus for measuring mercury in coal combustion exhaust gas by using the same and in particular to a method and apparatus for measuring total mercury with undergoing less influence of coexisting components interfering with total-mercury measurement, such as gasified metal oxides and sulfur dioxide (SO₂) in coal combustion exhaust gas from fossil fuel facilities, particularly from coal fuel facilities.

2. Description of the Related Art

As an apparatus for measuring total metal mercury in combustion exhaust gas, there has been conventionally used an apparatus for measuring total mercury for a fixed source by using a continuous measurement method or a dilution measurement method of using a gold amalgam catching/concentrating operation, stipulated under JIS K 0222. The dilution measurement method of using gold amalgam is a method of measuring metal mercury, which comprises heating a sample gas at high temperatures to reduce a mercury compound into metal mercury, then diluting it to catch mercury as gold amalgam, and after a predetermined time, re-gasifying amalgam mercury at high temperatures, and measuring metal mercury by a ultraviolet absorption method (see for example JIS K 0222-1997).

As applications are expanded in recent years, however, conventional methods of measuring mercury in, for example, exhaust gas from combustors are influenced by the presence of nitrogen oxides (NOx), sulfur dioxide (SO₂) or hydrogen chloride (HCl) in the exhaust gas, and thus it is difficult to obtain sufficiently accurate measurement values. At the request of improvement of measurement methods or of new measurement methods, the following various proposals are made at present.

Specifically, as shown in FIG. 7, there is proposed a method of continuously analyzing gaseous total mercury contained in exhaust gas upon treatment of sludge and wastes, wherein a mercury-containing gas is heated (about 230° C.) if necessary and then the mercury-containing gas is treated in a gaseous form with a heated (about 200° C.) solid reduction catalyst 21 consisting of a metal (metal tin, metal zinc etc.) thereby reducing a mercury compound (mercury chloride, mercury oxide etc.) in the mercury-containing gas into metal mercury which is then measured with a flameless atomic absorption spectrometer 22 (see, for example, JP-B 1-54655).

In an apparatus 31 for analyzing mercury in a mercuric chloride-containing gas, as shown in FIGS. 8(A) and (B), a reducing agent 34 comprising a stannous chloride coating 33 formed on the surface of tin particle 32 is charged into a reduction reactor 35, and by a reduction apparatus 36, the gas is passed through the reduction reactor 35, whereby Hg²⁺ in mercuric chloride is reduced to Hg⁰ by the reducing agent 34, and the reduced Hg⁰ is analyzed by an analyzer (flameless atomic absorption spectrometer) 37. By doing so, mercury analysis can be properly carried out even if the concentration of mercuric chloride in the gas is low (see, for example, JP-A 2001-33434).

However, when the measurement methods or measuring apparatuses described above are used to measure total mercury in coal combustion exhaust gas, accurate measurement is difficult because of poisoning of the catalyst by metal oxides such as selenium oxide and arsenic oxide (both of which are gases) coexisting in exhaust gas and the influence of coexisting gas components SO₂, NO₂ and water on the catalytic activity.

That is, it follows that in the atomic absorption spectrometry, photoabsorption in the ultraviolet range is utilized, and thus the interference influence of SO₂ and NO₂ coexistent at a high concentration of several thousand ppm in coal combustion exhaust gas cannot be negligible.

The dilution measurement method of using a gold amalgam catching/concentrating operation prescribed in JIS K 0222 supra has problems such as significant errors in dilution, limitation to batch measurement, and deterioration in performance of high-temperature reduction catalyst. This conventional method makes use of a high-temperature catalyst, but there is also a problem of necessity for arrangement of an acid scrubber because SO₂ is oxidized at high temperatures to form SO₃ mist. Further, element mercury is easily oxidized again with gas-contacting materials (for example, stainless steel (SUS)) used for the high-temperature catalyst, so the selection of a material constituting the catalyst unit is necessary.

Particularly in long-term use, removal of SeO₂ is essential, but a method therefore has not been established until now, and there is severe demand for the efficiency of removal thereof. That is, when a remover is not arranged, brownish-red element Se is formed uniformly over an inner surface of a piping under conditions for mercury-reducing conditions to occur at higher concentration on the surface where the flow rate of a gas is relatively low. As a result of verification with a mercury measuring instrument, the measured value of Hg was gradually decreased and sometimes reduced even by half in one week or so. This tendency was further significant where the measurement concentration was in the vicinity of a concentration as very low as 10 μg/m³. That is, SeO₂, even in a trace amount, forms amalgam gradually to often increase its influence, and a means of removing SeO₂ with high removal efficiency of not 90% or so but 95% or more has been required for the sample treatment system durable for such long-term use.

A gold-amalgam dilution measurement method as prescribed in JIS K 0222 suffers from problems such as significant errors in dilution, limitation to batch measurement, and deterioration in performance of high-temperature reduction catalyst. Specifically, there are problems (a) easy re-oxidation of mercury due to high-temperature deterioration of a catalyst material, dust adhesion, and corrosion of a gas-contacting material, and (b) inferior maintenance performance because of necessity for an acid scrubber against generation of an adhering component from mists of coexisting oxidized SO₂.

In spite of the demand mentioned above, an apparatus for continuously measuring mercury by an extraction sampling system directed to coal combustion exhaust gas and other than the dilution method.

### SUMMARY OF THE INVENTION

To cope with such demand, the object of the invention is to provide a method and an apparatus for removing selenium oxide (SeO₂) in coal combustion exhaust gas by easy operation stably for a long time in order to prevent the formation of element Se from SeO₂, which is interfering with the measurement of mercury in the exhaust gas. The present invention also provides a method and apparatus capable of continuously measuring mercury in coal combustion exhaust gas highly accurately with high long-term stability without undergoing the influence of coexisting components by using the removing method and removing apparatus described above.

The present inventors made extensive study, and as a result they found that the above object can be achieved by a method and apparatus for removing selenium oxide in a sample as well as a method and apparatus for measuring mercury in coal combustion exhaust gas by using the same, and the present invention was thereby completed.

That is, the present invention relates to a method of removing selenium oxide in a sample, comprising:

(1) heat treatment of a sample,
(2) primary cooling treatment wherein the sample in a high-temperature state is cooled by mixing with cooling water,
(3) secondary cooling treatment wherein the mixed gas is subjected to gas/liquid separation and simultaneously further cooled,
(4) regeneration of condensed water recovered by the secondary cooling treatment, and
(5) reutilizing the condensed water by cycling as cooling water in the primary cooling treatment.

The present invention also relates to an apparatus for removing selenium oxide in a sample, comprising:

(1) a heating introduction path for heating a sample,
(2) a primary cooling unit having a flow path through which the heated sample flows countercurrently to cooling water, whereby the heated sample is mixed with, and cooled by, cooling water,
(3) a secondary cooling unit having a spiral flow path for cooling the mixed gas and having a space for gas/liquid separation at the end of the spiral flow path,
(4) a regenerator for introducing condensed water from the secondary cooling unit, and
(5) a condensed water-cooling path for connecting the regenerator to the primary cooling unit.

As described above, it was found that in measurement of mercury in exhaust gas, SeO₂ present in the sample forms amalgam with mercury easily during reduction reaction, which is a major cause for significant deterioration in measurement accuracy. That is, SeO₂ forms selenious acid (H₂SeO₃) in the presence of water, as shown in reaction 1 below, and then H₂SeO₃ reacts with coexistent SO₂ or NO₂ to form an element Se, as shown in reaction 2 below. It was found particularly through the inventors' verification that the reaction proceeds more rapidly at higher temperature in the presence of water. The element Se forms amalgam with mercury (Hg) as shown in reaction 3 below. At this time, the element Se adheres as solidified matter to the flow path, thereby accelerating formation of mercury amalgam, resulting in further influence on measurement accuracy.

SeO₂+H₂O→H₂SeO₃ (Reaction 1)

H₂SeO₃+SO₂→Se+H₂SO₄ (Reaction 2)

Hg+Se→HgSe (Reaction 3)

In the prior method, it is difficult to remove SeO₂ without influencing measurement of mercury. In the present invention, the method of selectively removing SeO₂ was verified to eliminate such influence, thereby attaining measurement accuracy not achievable by the prior method.

That is, an SeO₂-containing sample in a state heated at a temperature of 100 to 200° C. is cooled rapidly to ambient temperature (usually 0 to 30° C. or so), thereby accelerating dissolution of SeO₂ in condensed water generated from moisture in the sample, to enable promotion of the reaction 1 above. At this time, the condensed water in the form of droplets induces the reaction 2 by dissolving SO₂ or NO₂ therein, but is thus washed away from the flow path by supplying of cooling water, whereby the reaction 2 can be suppressed. Supply of cooling water permits promotion of dissolution of H₂SeO₃ in the cooling water and simultaneously achieves an effect of diluting dissolved H₂SeO₃ and SO₂. Furthermore by lowering the temperature with cooling water, the reaction 3 can be lowered. In the present invention, it was found through verification that such technical effects can be practically achieved with the flow path through which a heated sample flows countercurrently to cooling water whereby the heated sample can be mixed with, and cooled rapidly by, cooling water.

In the present invention, such mixed gas can be cooled in a spiral flow path and simultaneously subjected to gas/liquid separation, thereby eliminating formation of amalgam and forming a sample gas from which SeO₂ was removed. That is, the spiral narrow flow path can be cooled whereby droplet generation, and splashing, in the flow path accompanying transfer of the mixed gas and generation of condensed water can be prevented, then the gas/liquid separation treatment can be effectively conducted in a space arranged at the end of the spiral flow path.

It is also preferable from the viewpoint of resource saving and energy saving or of decrease in the burden of wastewater treatment that condensed water obtained by the gas/liquid separation treatment is used as cooling water to be fed to the primary cooling unit, without externally continuously feeding cooling water for the primary cooling treatment. That is, water-soluble substances (for example, SeO₂ etc.) contained in a sample are in minute amounts, and selenious acid etc. can be easily removed by passing the condensed water through a regeneration means such as ion-exchange resin. Furthermore, when a coal combustion exhaust gas is used as a sample, the sample contains a large amount of moisture and does not need supply of cooling water, and therefore, such reutilization by cycling is also preferable for long-term use.

By the structure described above, there can be provided a method and apparatus for removing SeO₂ in a sample stably for a long time by easy operation.

In place of the combination of the primary cooling unit and the secondary cooling unit, it is possible to use a cooling treatment unit having (a) a water feed opening for the cooling water upstream of a spiral flow path, (b) a feed opening of the sample downstream of the water feed opening, (c) a space for gas/liquid separation, arranged at the end of the spiral flow path, (d) a condensed-water discharge flow path and a treated-gas feed flow path, branched with the space, and (e) a cooling means for cooling each of the path flows and the space.

One feature of the present invention in eliminating the influence of SeO₂ in a sample is that the gas/liquid is contacted with cooling water under such temperature conditions as not to generate droplets, and fundamentally a combination of the primary cooling treatment and secondary cooling treatment is preferable. However, the sample that is a gas has low heat capacity, while cooling water has high heat capacity and is capable of cooling to nearly 0° C., so that when the amount of the sample to be treated is relatively small, the primary cooling treatment and secondary cooling treatment can also be simultaneously conducted. In the present invention, downsized efficient cooling treatment was realized not only by these functions, but also by feeding cooling water from the uppermost stream, by high efficiency of heat exchange of the spiral flow path, and by upon jetting out from the narrow flow path to the expanded space, to prevent droplets and spray from being incorporated into the treated-gas feed flow path during liquid/gas separation.

The present invention relates to a method of removing selenium oxide in the sample, which comprises passing the sample under heating conditions through a scrubber charged with a barium compound or an iron oxide, or a mixture thereof, thereby selectively removing selenium oxide.

The present invention relates to an apparatus of removing selenium oxide in the sample, which comprises an introduction path for heating the sample, a scrubber charged with a barium compound or an iron oxide, or a mixture thereof, and a heating means for keeping the scrubber at a predetermined temperature, in order to selectively remove selenium oxide.

As described above, when a sample is treated with cooling water, water-soluble measurement components when contained in the sample may be dissolved in cooling water to cause a measurement error. For example, when a coal combustion exhaust gas is treated as a sample, mercuric chloride (Hg²⁺) is partially dissolved in cooling water and should thus be reduced into metal mercury (Hg⁰) prior to treatment, to make the sample treatment restrictive, and selective removal of selenium oxide under dry conditions becomes necessary. At this time, it is difficult to remove SeO₂ without influencing measurement of mercury, and there is no prior effective method. The present inventors verified the method of selectively removing SeO₂ with various metal compounds, and as a result, they found that a barium compound or an iron oxide can react specifically with SeO₂ as shown in reactions 4 and 5 below, under established conditions where there is little influence of mercury reaction or adsorption.

SeO₂+BaCO₃→BaSeO₃+CO₂ (Reaction 4)

xSeO₂+yFeO→FexSey+(x+y/2)O₂ (Reaction 5)

Accordingly, even in the case of a sample wherein a water-soluble measurement component is coexistent with SeO₂, the sample can be treated with the above compound as a scrubber to remove SeO₂ selectively under dry conditions, whereby the measurement accuracy of the measurement component can be secured.

The present invention relates to the method of removing selenium oxide in the sample, wherein a combination of the primary cooling treatment and the secondary cooling treatment, and the treatment for selective removal of selenium oxide, are carried out in series or in parallel.

The present invention also relates to the apparatus for removing selenium oxide in the sample, wherein a combination of the primary cooling unit and the secondary cooling unit, or the cooling treatment unit, and the scrubber are arranged in series or in parallel.

With respect to removal of SeO₂ in a sample, two effective methods, that is, treatment by a combination of the primary cooling treatment and secondary cooling treatment described above under wet conditions (referred to hereinafter as “wet treatment”) and treatment with a scrubber under dry conditions (referred to hereinafter as “dry treatment”), were found as a result of verification. The respective methods were found to secure 95% or more efficiency of removal as described later and each have unique advantages, although predetermined maintenance may be needed. That is, the wet treatment even when used for a long time can maintain the efficiency of removal, although the efficiency of removal may be lower than by the dry treatment. Further, the sample treatment method may be limited depending on coexistent components in a sample. The dry treatment can secure high selectivity and efficiency of removal, although a barium compound or an iron oxide used as the scrubber is consumed by the reaction, and thus there is a limit to its usable time. The present invention contemplates using the two methods complementarily by combining the two in series or in parallel.

Although the influence of the unremoved component even in an amount of 1% or less may gradually augment specifically in long-term use, a sample treatment system durable for such long-term use can be provided by combining the two methods in series and using the two complementarily. That is, when the dry treatment is arranged downstream of the wet treatment, a trace amount of SeO₂ remaining in the wet-treated sample can be decreased to an ultratrace amount by the dry treatment. In a coal combustion boiler, large amounts of mercury and SeO₂ may be contained in a sample in the start-up boiler, and during steady operation, these may be decreased in trace amounts. In such cases, the wet treatment and the dry treatment are arranged in parallel, and the sample is subjected to wet treatment in the former and to dry treatment in the latter, whereby both of the treatments can work complementarily to decrease the loading on each treatment.

The present invention relates to a method of measuring mercury in coal combustion exhaust gas by using the method or apparatus for removing selenium oxide in a sample, which comprises treating, by the removing method or with the removing apparatus, a coal combustion exhaust gas as a measurement sample collected from a sampling part and measuring it with a mercury analyzer.

The present invention also relates to an apparatus for measuring mercury in coal combustion exhaust gas by using the method or apparatus for removing selenium oxide in a sample, which comprises a sampling part for collecting a coal combustion exhaust gas as the sample, a sample introducing path for heating and introducing the sample from the sampling part, the removing apparatus, and a mercury analyzer.

It was found that in measurement of mercury in exhaust gas, a mercury compound is reduced into atomic mercury which is then measured by absorption spectroscopy thereby achieving extremely highly sensitive measurement, while in measurement of mercury in coal combustion exhaust gas, some new problems should be overcome. Particularly SeO₂ present in exhaust gas easily forms amalgam with mercury during reduction reaction and is thus one of major causes for significant deterioration in measurement accuracy, and the present invention uses the method or apparatus for removing selenium oxide in a sample to eliminate its influence thereby securing measurement accuracy not achievable by the prior art. Accordingly, it became possible to provide a method and apparatus for measuring mercury in coal combustion exhaust gas, which can continuously measure mercury highly accurately, and stably for a long time without undergoing the influence of the coexisting components.

The present invention relates to a method of measuring mercury in coal combustion exhaust gas, wherein the sample is treated by the removing method or with the removing apparatus above described, and then the sample is measured with an ultraviolet absorption analyzer by comparison with a reduced gas, wherein mercury in the sample was reduced with a catalyst consisting of an inorganic material having reducing power, and an oxidized gas, wherein the measurement sample or the sample gas was oxidized with an oxidation catalyst.

The present invention relates to the apparatus for measuring mercury in coal combustion exhaust gas, comprising a sample introducing path for heating and introducing the sample from the removing apparatus above described, a reduction catalyst part charged with a catalyst of an inorganic material having reducing power toward mercury and being poor in reactivity with acidic substances, a reduced-gas flow path provided with the reduction catalyst part, an oxidation catalyst part charged with an oxidation catalyst, an oxidized-gas flow path provided with the oxidation catalyst part, and an ultraviolet absorption analyzer for measuring mercury concentration by comparison between the reduced gas and the oxidized gas.

In measurement of mercury in coal combustion exhaust gas, there are some problems to be solved along with treatment of SeO₂ present in the exhaust gas. That is, in coal combustion exhaust gas, mercury occurs in the state of Hg²⁺ or Hg^{o}, and simultaneously components such as SO₂, NO₂ and moisture, causing measurement errors such as interference influence on an ultraviolet absorption analyzer are also coexisting. In the present invention, a reduced gas wherein total mercury is converted into Hg^{o} by selectively reducing the mercury in the sample, and an oxidized gas wherein total mercury is converted into Hg²⁺ by selectively oxidizing the mercury in the sample, are prepared, and

(1) when there is a single ultraviolet absorption cell (sample cell) in an ultraviolet absorption analyzer, the reduced gas and oxidized gas are introduced alternately into the sample cell, and both of them are compared with respect to the quantity of absorption light, or
(2) when there are plural (usually two) sample cells, the reduced gas and oxidized gas are introduced simultaneously into the sample cells respectively, and both of them are measured for their difference in the quantity of absorption light,

thereby enabling measurement without undergoing the influence of other coexistent components not changed by both oxidation treatment and reduction treatment.

That is, one sample is subjected in series or in parallel to oxidation and reduction, and two samples obtained by oxidation and reduction respectively are measured for their difference in the state of mercury therein, whereby high selectivity and measurement accuracy in measurement of mercury in the coal exhaust gas can be secured.

Specifically, a catalyst of an inorganic material having reducing power and being poor in reactivity with acidic substances can be used to eliminate the poisoning effect, on the catalyst itself, of acidic substances such as SO₂ and NO₂ contained in large amounts in coat combustion exhaust gas. By introducing the samples thus treated into an ultraviolet absorption analyzer, analytical functions similar to those of the atomic absorption method can be secured to enable highly accurate measurement of mercury.

The “catalyst of an inorganic material having reducing power toward mercury and being poor in reactivity with acidic substances”, as used herein, refers to catalysts such as zeolite-based catalysts described later or to inorganic compounds such as alkali metal sulfites, serving as catalysts which have a function to reduce divalent-mercury (Hg²⁺) compounds such as mercury chloride (HgCl₂) into the metal (Hg^{o}) and are poor in reactivity with acidic substances such as SO₂ and NO₂ contained in large amounts in coal combustion exhaust gas.

According to the present invention, there can be provided a method and an apparatus for removing selenium oxide (SeO₂) in coal combustion exhaust gas by easy operation stably for a long time in order to prevent the formation of element Se from SeO₂, which is interfering with the measurement of mercury in the exhaust gas and is conventionally difficult to be prevented. In addition, a method and apparatus capable of continuously measuring mercury in coal combustion exhaust gas highly accurately with high long-term stability without undergoing the influence of coexisting components can be provided by using the removing method and removing apparatus described above.

Particularly, higher accurate measurement without undergoing the influence of coexistent components can be made feasible by using a combination of the reduction and oxidization catalyst parts to compare and measure the respective treated gases.

### BRIEF DESCRIPTION OF THE DRAWINGS

FIG. 1 is an illustration showing a first constitutional example of the apparatus for removing selenium oxide according to the present invention;

FIG. 2 is an illustration schematically showing a primary cooling pipe used in the sample treatment system according to the present invention;

FIG. 3 is an illustration schematically showing a secondary cooling pipe used in the sample treatment system according to the present invention;

FIG. 4 is an illustration schematically showing a cooling treatment unit used in the sample treatment system according to the present invention;

FIG. 5 is an illustration showing an applied example of the first constitutional example of the apparatus for removing selenium oxide according to the present invention;

FIG. 6 is an illustration showing a second constitutional example of the apparatus for removing selenium oxide according to the present invention;

FIG. 7 is an illustration schematically showing the test apparatus for removing selenium oxide according to the present invention;

FIG. 8 is an illustration showing a third constitutional example of the apparatus for removing selenium oxide according to the present invention;

FIG. 9 is an illustration showing another third constitutional example of the apparatus for removing selenium oxide according to the present invention;

FIG. 10 is an illustration showing one constitutional example of the apparatus for measuring mercury according to the present invention;

FIG. 11 is an illustration showing another constitutional example of the apparatus for measuring mercury according to the present invention;

FIG. 12 is an illustration showing a constitution of an analyzer according to the prior art; and

FIG. 13 is an illustration showing a constitution of another analyzer according to the prior art.

In the illustrations, 1 is a heating conduit; 1a, a sample feed path; 2, a primary cooling pipe; 3, a secondary cooling pipe; 3a, electron cooler; 4, a regenerator; 5a, a drain recovery pump; 5b, a cooling-water tank; 5c, a cooling-water feed pump; 5d, a flowmeter; 7, a scrubber; 10, an ultraviolet absorption analyzer; 11, a sample inlet; 12, a dust filter; 13, a reducing catalyst part; 14, a filter; and 15, a suction pump.

### DETAILED DESCRIPTION OF THE INVENTION

Hereinafter, embodiments of the present invention are described in detail by reference to drawings.

### <First Constitutional Example of the Removing Apparatus>

In one embodiment of the present invention, the apparatus for removing selenium oxide (referred to hereinafter as “the present removing apparatus”) comprises:

(1) a heating introduction path for heating a sample,
(2) a primary cooling unit having a flow path through which the heated sample flows countercurrently to cooling water, whereby the heated sample is mixed with, and cooled by, cooling water,
(3) a secondary cooling unit having a spiral flow path for cooling the mixed gas and having a space for gas/liquid separation at the end of the spiral flow path,
(4) a regenerator into which condensed water from the secondary cooling unit is introduced, and
(5) a cooling-water feed path for connecting the regenerator to the primary cooling unit.

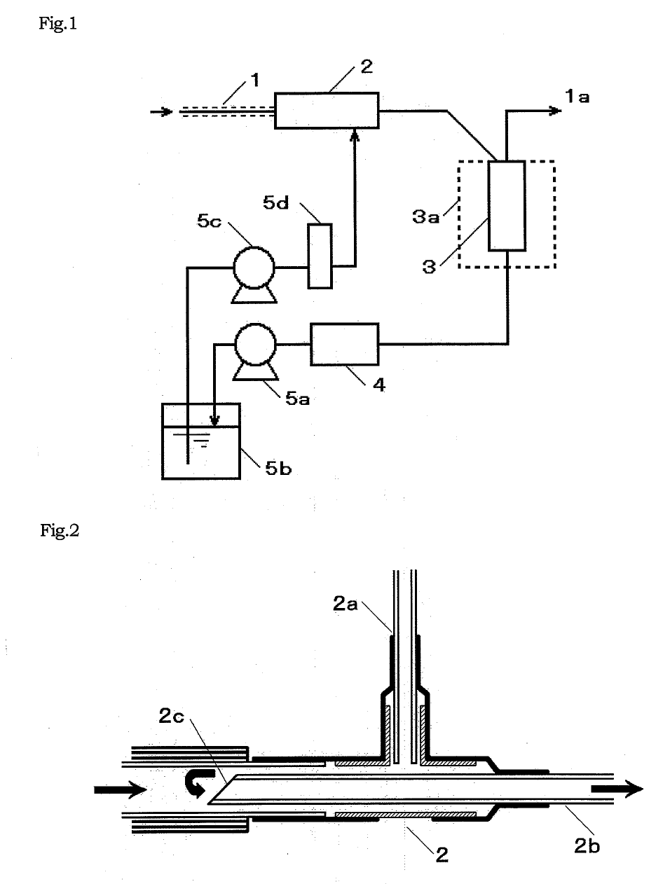

A specific first constitution of the present removing apparatus is illustrated in FIG. 1 (first constitutional example). The removing apparatus is composed of a heating conduit 1 (corresponding to a heating introduction path), a primary cooling pipe 2 (corresponding to a primary cooling unit), a secondary cooling pipe 3 and an electron cooler 3a (corresponding to a secondary cooling unit) for cooling the pipe 3, a regenerator 4 charged with an anion exchanger, a drain recovery pump 5a, a cooling-water tank 5b, a cooling-water feed pump 5c and a flowmeter 5d (forming a cooling-water feed path).

A sample containing moisture, SeO₂ etc., while being heated with the heating conduit 1 so as not to be condensed, is transferred and introduced into the primary cooling pipe 2 and mixed with cooling water. In the primary cooling pipe 2, the sample is rapidly cooled, while SeO₂ in the sample is dissolved in the cooling water, and then introduced into the secondary cooling pipe 3. In the secondary cooling pipe 3, the sample is further cooled, subjected to gas/liquid separation and fed as a treated dry sample via a sample feed path 1a from an upper part of the secondary cooling pipe 3.

On the other hand, cooling water stored in the cooling-water tank 5b is fed via the flowmeter 5d with the cooling-water feed pump 5c to the primary cooling pipe 2. In the primary cooling pipe 2, the sample is cooled (while cooling water is warmed), and simultaneously SeO₂ in the sample is removed by dissolution and introduced together with the sample into the secondary cooling pipe 3. In the secondary cooling pipe 3, the sample is cooled with the electron cooler 3a and simultaneously subjected to gas/liquid separation, suctioned with the drain recovery pump 5a, and recovered in the cooling water tank 5b via the regenerator 4 charged with an anion exchanger. In the regenerator 4, SeO₂ and other water-soluble substances dissolved in cooling water are removed, whereby the cooling water is recovered as clean cooling water.

The mechanism of dissolution of SeO₂ in condensed water and formation of amalgam with mercury is described, and the role of each component is described.

Mechanism of Dissolution of SeO₂ in Condensed Water and Formation of Amalgam with Mercury

(a) As shown in reaction 1 below, SeO₂ is soluble in water to form H₂SeO₃. As a result of verification, this reaction was found to proceed very rapidly.

SeO₂+H₂O→H₂SeO₃ (Reaction 1)

(b) As shown in reaction 2 or 2′ below, the formed H₂SeO₃ is reduced with a large amount of coexistent SO₂ in exhaust gas to form metal Se. As a result of verification, this reaction was found to proceed relatively moderately and does not occur just after SeO₂ is dissolved in water. This was experimentally proven by the fact that when a predetermined concentration of SO₂ gas is introduced into an aqueous solution of H₂SeO₃, the solution turns yellow to orange, to gradually form precipitates of dark orange (selenium (Se) element). Se is formed rapidly in a high-temperature and a high-dew-point atmosphere, and Se once precipitated to adhere to a pipe wall cannot be expected to be washable with water.

H₂SeO₃+SO₂→Se+H₂SO₄ (Reaction 2)

H₂SeO₃+2SO₂+H₂O→Se+2H₂SO₄ (Reaction 2′)

(c) Generated metal Se is water-insoluble and is precipitated as red powdery matter on an internal wall etc. of the pipe to easily form amalgam with mercury, as shown in the following reaction 3:

Hg+Se→HgSe (Reaction 3)

Constitution of the Present Removing Apparatus

### (1) Heating Introduction Path (Heating Conduit 1)

A sample is heated at 100 to 200° C. Condensation of moisture in the sample can thereby be suppressed, and formation of selenious acid (H₂SeO₃) by the above reaction 1 can be suppressed. The generation of element selenium by the above reaction 2 or 2′ of selenious acid with SO₂ or NO₂ in the sample in the presence of water (including droplets and spray) is suppressed and consequently the above reaction 3 is suppressed, whereby the reaction of mercury in the sample with Se can be suppressed.

(2) Primary Cooling Unit (Primary Cooling Pipe 2)

The sample heated at 100 to 200° C. is cooled rapidly to ambient temperature with the primary cooling pipe 2 and simultaneously mixed with cooling water. SeO₂ in the sample is dissolved in cooling water by the above reaction 1 and then washed away with cooling water such that selenious acid generated by the reaction 1 does not remain on a flow path of the cooling pipe etc. The primary cooling pipe 2 is not limited with respect to its structure and material insofar as the pipe has the above function and is corrosion-resistant. For example, the structure shown in FIG. 2 is preferable. The primary cooling pipe 2 is in the form of a T-tube with a cooling water inlet 2a as the top, having a narrow pipe (sample-conduit) 2b such as 3φ/2φ fluorine resin pipe inserted into it. Cooling water introduced into the primary cooling pipe 2 flows from the cooling water inlet 2a via an obliquely cut part 2c arranged in the outlet of the heating conduit 1 to the sample-conduit 2b, to accelerate cooling of the sample gas and dissolution of sample gas SeO₂ in cooling water. With this structure given, rapid cooling can be maintained stably for a long time. The sample-conduit 2b is connected to the secondary cooling pipe 3, and the cooling water containing formed selenious acid dissolved therein, and a mixed gas of the sample, are fed from the sample-conduit 2b to the secondary cooling pipe 3.

(3) Secondary Cooling Unit (The Secondary Cooling Pipe 3 and the Electron Cooler 3a for Cooling it)

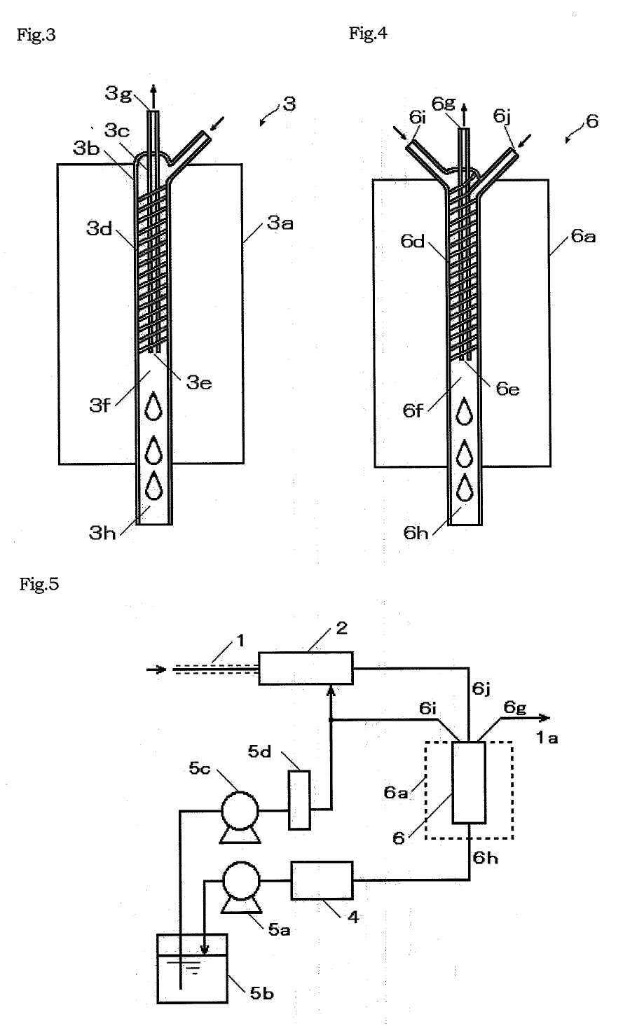

The secondary cooling pipe 3 is not particularly limited with respect to its structure and material insofar as it can efficiently cool a gas/liquid mixed fluid consisting of cooling water and condensed water (referred to hereinafter as “cooling water etc.”) and the sample, can increase the exit velocity to contribute to an effect of washing the inside of the cooling pipe, and is corrosion-resistant. For example, the structure shown in FIG. 3 is preferable. The secondary cooling pipe 3 arranged in the electron cooler 3a or in a water-cooling cooler has a double-pipe structure using a glass pipe, consisting of a spiral flow path 3d arranged between an outer pipe 3b contacting with a heat-exchange part of the electron cooler 3a and an inner pipe 3c, and a space 3f arranged in the end 3e thereof, and cools the sample and increases the outflow of cooling water etc. The sample that has passed through the flow path 3d is separated from cooling water etc. in the lower space 3f, and passes through an inner flow path 3g in an inner pipe 3c. During this time, the cooled sample is subjected to heat exchange with the sample passing through the flow path 3d. Although significantly efficient heat exchange cannot be expected, the outgoing low-temperature sample is re-heated by heat exchange with the introduced high-temperature sample, thereby preventing dew formation. By treating the sample in such secondary cooling pipe 3, formation of interfering element Se can be prevented. On the other hand, cooling water etc. are reused by cycling through a condensed-water discharge path 3h from the space 3f, and by rapidly cooling the sample with the primary cooling pipe 2 and adding cooling water to the sample, the flow rate in the drain is increased, and while the sample always flows in the sample treatment system, the generated drain flow is discharged out of the system. When a naturally dropped drained waste not reutilized by cycling is retained in a pot, formation of element Se may occurs not only in the drain flow path but also in a sample flow path connected thereto.

(3′) Cooling Treatment Unit 6

In place of a combination of the primary cooling pipe 2 and secondary cooling pipe 3, a structure wherein a water feed opening 6i for cooling water may be arranged upstream of the spiral flow path 6d as shown in FIG. 4 to feed cooling water therethrough can be used (referred to hereinafter as “cooling treatment unit 6”). The cooling treatment unit 6 composed of a sample feed opening 6j downstream of the water feed opening 6i, a space 6f for gas/liquid separation arranged in the end 6e of the flow path 6d, a condensed-water discharge flow path 6h and treated-gas feed flow path 6g branched with the space 6f, and the electron cooler 6a for cooling each flow path and the space 6f, can be used to achieve the same effect as described above and to downsize the cooling treatment unit 6. By adding moisture by injection of cooling water during both measurement of sample gas and checking with calibration gas, both the saturated dilution ratio of moisture and the influence of interfering moisture can be corrected to make highly accurate calibration feasible.

(4) Regenerator 4

Cooling water etc. dropped from the condensed-water discharge flow path 6h of the secondary cooling pipe 3 is regenerated with a regenerator 4 and reutilized as cooling water by cycling. The regenerator 4 is charged with a reagent for removing interfering selenious acid in cooling water etc. Specifically, anion-exchange resin and selenious-acid adsorbents such as iron oxides (for example ferrous oxide (FeO) and iron oxyhydroxide (FeO.OH)) can be used, among which anion-exchange resin that can be regenerated is preferable. In the present apparatus, the regenerator is charged with about 250 g anion-exchange resin through which cooling water etc. pass constantly at a rate of 1 to 10 ml/min. As a result of verification of the frequency of maintenance such as exchange and replenishment of cooling water, replenishment is conventionally necessary per month, but it was found that when anion-exchange resin is used, replenishment may be carried out only once for 3 to 6 months. Unlike FIG. 1, the generator 4 may be arranged not downstream of the secondary cooling unit 3 but before or after the cooling water feed pump 5c.

(5) Cooling Water Feed Path (The Drain Recovery pump 5a, the Cooling Water Tank 5b, the Cooling Water Feed Pump 5c and the Flowmeter 5d)

Cooling water etc. dropped from the condensed water discharge flow path 6h of the secondary cooling pipe 3 passes through the regenerator 4, the drain recovery pump 5a, the cooling water tank 5b, the cooling water feed pump 5c and the flowmeter 5d, and is introduced constantly as cooling water at a rate of about 1 to 10 ml/min. into the primary cooling pipe 2, whereby the cooling water is reutilized by cycling. As the drain recovery pump 5a and the cooling water feed pump 5c, tubing pumps are generally used for recovering and feeding an almost constant amount of liquid, but a decrease in the elasticity of the tube can cause a drop in flow rate, and thus the flowmeter 5d is preferably arranged in the outlet of the cooling water feed pump 5c as shown in FIG. 1, to monitor and periodically correct the flow rate. Arrangement of the flowmeter 5d for cycling cooling water is preferable because amalgam is formed when droplets are generated due to stopped feeding of cooling water to be reutilized by cycling. For monitoring the flow rate of cooling water etc. to be reutilized by cycling, a liquid level detector such as a float switch (not shown) can be used to detect an increase or decrease in a predetermined time in the amount of water in the cooling water tank 5b, and from the detected flow rate, the flow rate of cooling water fed can be corrected.

Example of Application of the Present Removing Apparatus

Depending on sample conditions, the present removing apparatus can be modified by replacement or detachment of the elements or by addition of other elements. For more effectively utilizing the cooling treatment unit 6, the structure illustrated in FIG. 5 can also be used. That is, cooling water stored in the cooling water tank 5b is branched via the cooling water feed pump 5c and flowmeter 5d and fed to the primary cooling pipe 2 and cooling treatment unit 6, while the primary cooling pipe 2 and cooling treatment unit 6 are arranged in series so that the sample is treated with cooling water in 2 stages, whereby formation of element Se can be prevented more efficiently.

Example of the Present Removing Apparatus

### (1) Experimental Conditions

An air containing 18 ppm SeO₂ was introduced at a flow rate of 1.1 L/min. downward from the heating conduit 1 of the present removing apparatus illustrated in FIG. 1.

### (2) Experimental Result

Cooling water recovered in the cooling water tank 5b was measured by the inductively coupled radio frequency plasma method (ICP, type: ULTIMA2, manufactured by Horiba, Ltd.), to obtain a concentration of 5 ppb of dissolved Se. From the amount of 300 g cooling water in the cycling system, the total amount of dissolved SeO₂ was calculated and the efficiency of removal was calculated to obtain a result of 95%.

Another Constitutional Example of the Present Removing Apparatus

Another removing apparatus of the present invention comprises:

(1) a heating introduction path for heating a sample,
(2) a scrubber charged with a barium compound or an iron oxide or a mixture thereof,
(3) a heating means for keeping the scrubber at a predetermined temperature,

wherein the treatment for selective removal of selenium oxide is carried out.

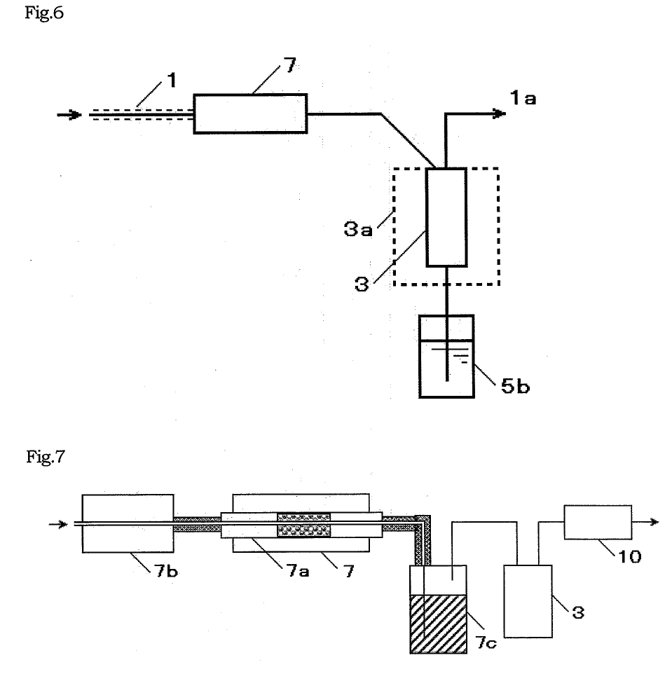

Another specific structure of the present removing apparatus is illustrated in FIG. 6 (second constitutional example). The apparatus is composed of a heating conduit 1 (corresponding to the heating introduction path), a scrubber 7 for removing SeO₂ heated with a heating means (not shown), a secondary cooling pipe 3 and an electron cooler 3a for cooling it, and a cooling water tank 5b.

A sample containing moisture, SeO₂ etc., while being heated with a heating conduit 1 so as not to be condensed, is transferred and introduced into a scrubber 7 heated at a predetermined temperature, to remove SeO₂ in the sample, and then introduced into a secondary cooling pipe 3. In the secondary cooling pipe 3, the sample is cooled, while generated condensed water is subjected to gas/liquid separation, and the sample passes from an upper part of the secondary cooling pipe 3 via a sample feed path la and is fed as a dry sample. On the other hand, the condensed water subjected to gas/liquid separation in the secondary cooling pipe 3 is stored in a cooling water tank 5b.

The scrubber 7 is a unit charged therein with an SeO₂ remover, wherein the SeO₂ remover is kept preferably at 150 to 250° C. by a heating means (not shown). That is, mercury etc. in coal combustion exhaust gas are easily adsorbed into the SeO₂ remover at a temperature of lower than 150° C., while the efficiency of reaction with SeO₂ (degree of removal of SeO₂) is decreased at a temperature of higher than 250° C., as described later, and thus the scrubber 7 is operated preferably in the above range.

Selection of SeO₂ Remover

(1) Verification with Various Metal Compounds

### (1-I) Experimental Conditions

Using the experimental apparatus illustrated in FIG. 7, a scrubber unit 7a was charged with various metal compounds usable as scrubber 7, and a testing gas was circulated at about 1.1 L/min. for 3 hours for testing. As the testing gas, an air containing SO₂ at a concentration of 500 ppm was introduced into an SeO₂ gasifying apparatus 7b set 200° C., to form a gas of SeO₂ at a concentration of 18 ppm. Similarly, a standard gas to generate mercury chloride (HgCl₂) whose concentration was previously determined was prepared (50 μg/m³). The heating temperature of the scrubber 7 was 150 to 250° C., and the gas that had passed through the scrubber was passed through an SeO₂ scavenger fluid 7c, and the amount of unremoved SeO₂ was examined. For determining the amount of unremoved SeO₂, the concentration of SeO₃ ion in the scavenger fluid was measured and the concentration of Se dissolved therein was analyzed. On the other hand, the HgCl₂-containing gas was dehumidified in the secondary cooling unit 3 and measured with an UV analyzer 10, to examine influence such as the presence or absence of absorption loss with the scrubber. For judging the degree of removal, the degree of occurrence of yellow to reddish brown precipitates on an outlet piping of the scrubber was also considered in addition to the degree of removal from the analytical value of SeO₃ ion concentration in the scavenger fluid.

### (1-2) Experimental Results

The test results are shown in Table 1. In the various metal compounds, a barium compound (barium carbonate (BaCO₃)) and iron(III) oxide (iron oxyhydroxide) gave excellent results satisfying conditions of the degree (99% or more) of removal of SeO₂ and the absence of adsorption of Hg(0) in the temperature range of about 200° C. The symbol “O” indicates a compound giving an excellent result, and “not removable” is given to a compound not capable of removal.

TABLE 1

| | Removal of | Removal of | Adsorption | SO₂ |
|---|---|---|---|---|
| | selenium | selenium | of Hg0 | mixture |
| Substance | (200° C.) | (380° C.) | (200° C.) | |
| | <Degree of | <Degree of | | <Degree of |
| | removal> | removal> | | removal> |
| BaCO₃ | ◯ | not | ◯ | ◯ |
| | | removable | | |
| | <99.6%> | | | <97.6%> |
| Iron | ◯ | not | ◯ | ◯ |
| oxyhydroxide | | removable | | |
| | <99.8%> | | | <97.9%> |

(2) Characteristics of Barium Compounds

As a result of the above verification, it was found that barium compounds such as barium carbonate (BaCO₃) and barium sulfite (BaSO₃) react selectively with SeO₂ as shown in reactions 6 and 7 below, under established conditions (temperature condition: 150 to 250° C.) where there is little influence of reaction with or adsorption of mercury.

SeO₂+BaCO₃→BaSeO₃+CO₂ (Reaction 6)

SeO₂+BaSO₃→BaSeO₃+SO₂ (Reaction 7)

As shown in Table 1 above, 99% or more removal can be secured at 200° C. Actually, moisture exists in coexistent gas and promotes the reaction and may partially form H₂SeO₃ to contribute to the reaction.

(3) Characteristics of Iron Oxides

It is estimated that iron oxides such as ferrous oxide (FeO) and iron oxyhydroxide (FeO.OH) react selectively with SeO₂ as shown in reaction 5 or in reactions 8 to 10 below, to form Fe₂(SeO₃)₃. At a temperature of 150 to 250° C., there was little influence of reaction with or adsorption of mercury. As shown in Table 1 above, 99% or more removal can be secured at 200° C.

xSeO₂+yFeO→FexSey+(x+y/2)O₂ (Reaction 5)

3SeO₂+2FeO+1/2O₂→Fe₂(SeO₃)₃ (Reaction 8)

3SeO₂+2FeO.OH→Fe₂(SeO₃)₃+H₂O (Reaction 9)

3SeO₂+Fe₂O₃→Fe₂(SeO₃)₃ (Reaction 10)

(4) Characteristics of Mixture

The selenium oxide remover reagent was exemplified by barium compounds and iron oxides, and a mixture thereof can be used to prolong longevity. The cause for deterioration in longevity is considered attributable to selenites (MSeO₃ and M₂(SeO₃)₃ etc. wherein M is Ba, Fe or the like) generated by the reaction, and when the removing scrubber reagent is used alone, a single selenite salt is generated, and when the single salt is formed on fine crystals of the reagent powder, the efficiency is caused to decrease. When the reagent is formed from a mixture of different reagents, a single salt is hardly formed as compared with the reagent composed of a single salt.

(5) Filler of the Scrubber

Any reagents mentioned above are powdery or fine crystalline reagents, but granular scrubbers formed from barium carbonate, iron oxides etc. are preferable for use as scrubber fillers. Using a binder solution, inorganic porous particles are granulated or formed into granules. Specifically, Pamister (trade name: manufactured by Ohe Kagaku Kogyo Co., Ltd.) or activated alumina is used as the inorganic porous particles, and water glass or lithium silicate is used as the binder. This filler can be arranged before the Hg reduction catalyst, to remove SeO₂ selectively without undergoing the influence of moisture and SO₂ in exhaust gas, thus enabling stable and highly accurate measurement of total mercury.

<Third Constitutional Example of the Present Removing Apparatus>

The third constitutional example of the present removing apparatus comprises a combination of a primary cooler and a secondary cooler or a cooling treatment unit arranged in series, or in parallel, with a scrubber. Although the wet treatment can maintain the efficiency of removal even in long-term use, the efficiency of removal may be lowered as compared with the dry treatment. Further, the sample treatment method may be limited depending on coexistent components in a sample. Although the dry treatment can secure high selectivity and efficiency of removal, the barium compound or iron oxide used as a scrubber is consumed by the reaction, and thus there is a limit to usable time. The present invention contemplates using the two methods complementarily by combining the two in series or in parallel.

(1) In the Case of Arrangement in Series

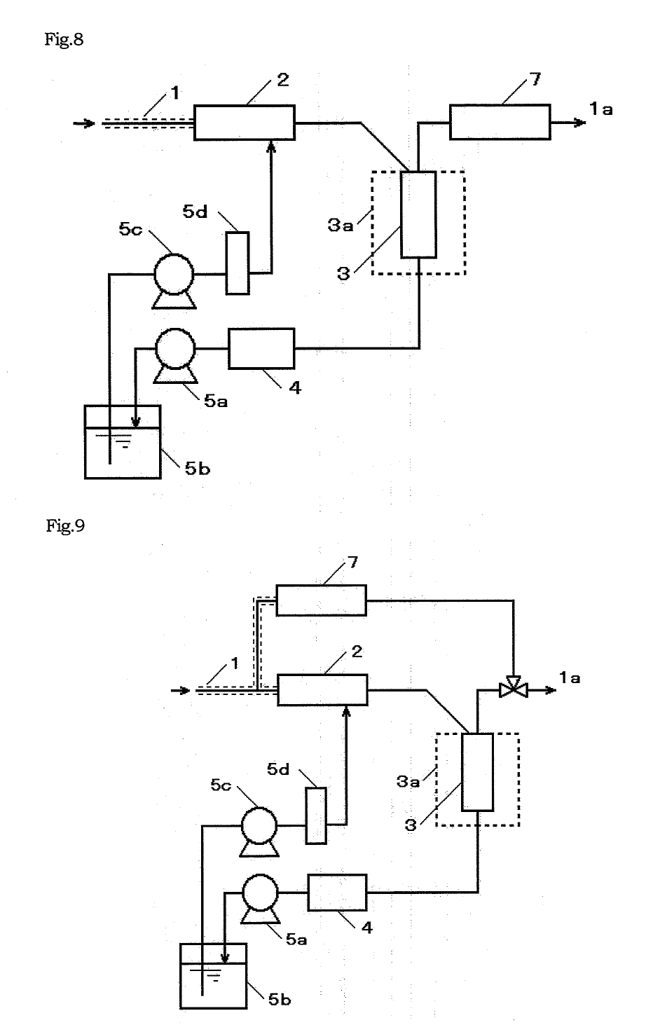

As illustrated in FIG. 8, a primary cooler 2 and a second cooler 3, and a scrubber 7 are arranged in series. A trace amount of SeO₂ remaining a sample treated with the primary cooler 2 and secondary cooler 3 can be decreased to the ultratrace level by the scrubber 7. The wet treatment is suitable for long-term use, and thus the primary cooler 2 and secondary cooler 3 can be arranged upstream to constitute a sample treatment system durable for long-term use.

(2) In the Case of Arrangement in Parallel

As illustrated in FIG. 9, a primary cooler 2, a second cooler 3 and a scrubber 7 are arranged in parallel. In a coal combustion boiler for example, large amounts of mercury and SeO₂ may be contained in a sample in the start-up boiler, and during steady operation, these may be decreased in trace amounts. In such cases, the wet treatment and dry treatment are arranged in parallel, and the sample is subjected to the wet treatment in the former and to the dry treatment in the latter, whereby both of the treatments can work complementarily to decrease the loading on each treatment.

Example of the Present Removing Apparatus

### (1) Experimental Conditions

An air containing 18 ppm SeO₂ was introduced at a flow rate of 1.1 L/min. downward from the heating conduit 1 of the present removing apparatus wherein the primary cooler 2, the second cooler 3 and the scrubber 7 were arranged in series, as illustrated in FIG. 8.

### (2) Experimental Result

Cooling water recovered in the cooling water tank 5b was measured by the inductively coupled radio frequency plasma method (ICP, type: ULTIMA2, manufactured by Horiba, Ltd.), to obtain a concentration of 5 ppb dissolved Se. From the amount of 300 g cooling water in the cycling system, the total amount of SeO₂ dissolved was calculated and the efficiency of removal was calculated to obtain a result of 95%.

<Constitutional Example of the Apparatus for Measuring Mercury in Coal Combustion Exhaust Gas by Using the Present Removing Apparatus>

The apparatus for measuring mercury in coal combustion exhaust gas (referred to hereinafter as “the present measuring apparatus”) by using the present removing apparatus measures a coal combustion exhaust gas as a measurement sample and comprises a sample collection part for collecting the sample, a sample introducing path for heating and introducing the sample from the sample collection part, the removing apparatus described above and a mercury analyzer. SeO₂ present in coal combustion exhaust gas easily forms amalgam with mercury in the coexistence of moisture, SO₂ and NO₂ contained in large amounts in the exhaust gas and is thus a major cause for significant deterioration in measurement accuracy, and the present measuring apparatus uses the present removing apparatus described above to eliminate this influence thereby securing measurement accuracy not attainable by the prior art.

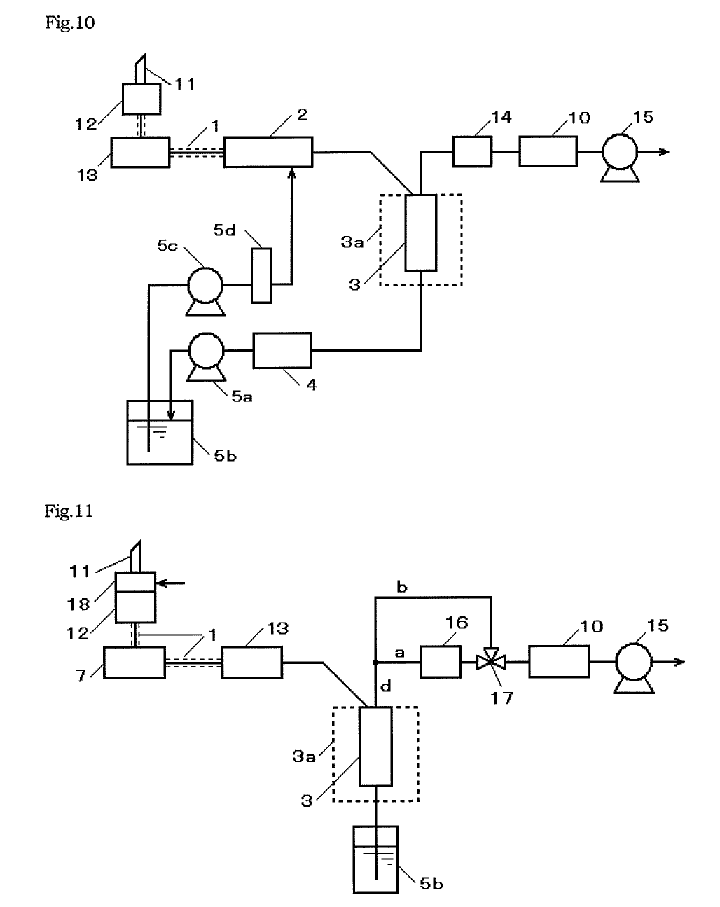

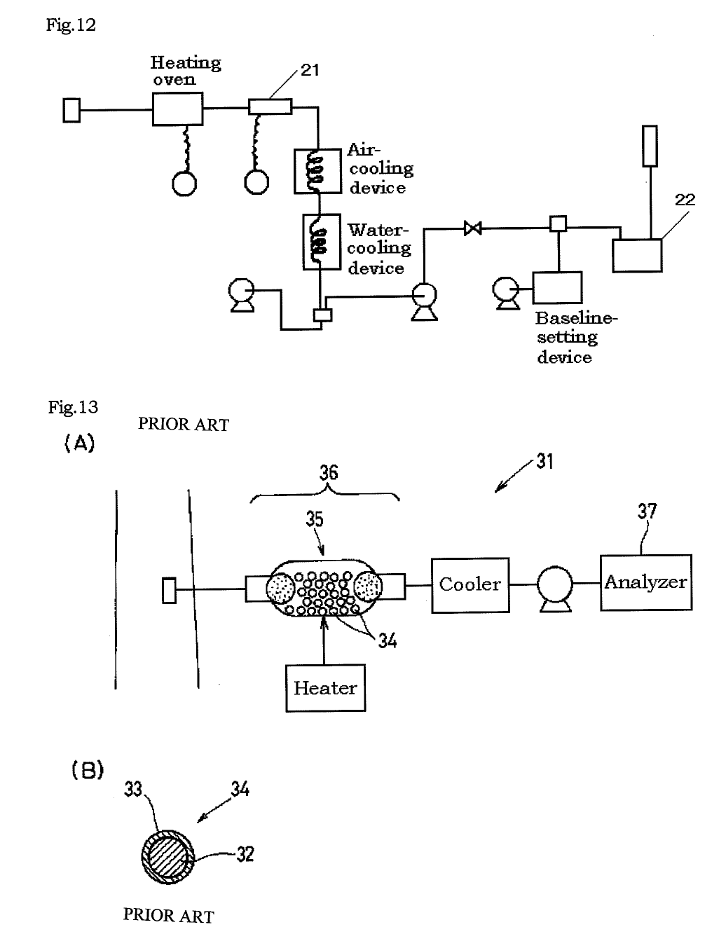

FIG. 10 illustrates one constitution of the present measuring apparatus. This constitution is suitable for measuring, in a sample, total mercury containing a plurality of mutually convertible components (Hg²⁺+Hg⁰) containing the same element, such as divalent mercury (Hg²⁺) and element mercury (Hg⁰) etc.. That is, Hg²⁺ in a sample gas is first converted into Hg0 as a measurement object, and then the sample is treated with the present removing apparatus to give a gas which can be analyzed without undergoing the influence of other coexistent components such as SeO₂.

Hereinafter, the apparatus for measuring total mercury in coal combustion exhaust gas according to the present invention, wherein the wet treatment is used in the present removing apparatus of the invention and the ultraviolet absorption analyzer 10 is used as a measuring means, is described in detail by reference to a specific example.

A sample is collected from a sample inlet 11 (corresponding to the sample collection part) by suction with a suction pump 15 arranged downstream of the ultraviolet absorption analyzer 10. The collected sample was cleaned with a dust filter 12, and then total mercury in the sample is converted into Hg⁰ with a reducing catalyst part 13, and introduced via a heating conduit 1, a primary cooler 2, a secondary cooler 3 and a filter 14 into the ultraviolet absorption analyzer 10. As the material contacting with the gas, it is possible to employ not only inexpensive glass, quartz, and ceramics but also metals such as Ti and anodized SUS.

The reducing catalyst part 13 is a unit charged therein with a reducing catalyst, wherein the reducing catalyst is kept preferably in the middle temperature range of 250 to 500° C. by a heating means (not shown). That is, mercury in coal combustion exhaust gas occurs in the form of HgO, HgCl₂ or Hg⁰. For conversion of Hg²⁺ into Hg⁰, pyrolysis reaction is essential, and when the reducing temperature is 250° C. or more, amalgam generated by the reaction of mercury with metal oxides such as SeO₂ contained in exhaust gas can be prevented from being generated. On the other hand, when the reducing temperature is 500° C. or less, troubles such as corrosion or reactant clogging in the sample flow path can be prevented.

The reducing catalyst charged into the reducing catalyst part 3 is preferably a catalyst of an inorganic material having reducing power and being poor in reactivity with acidic substances. In the present invention, it is required that the reducing catalyst has a function to reduce a divalent mercury (Hg²⁺) compound such as mercury chloride into a metal (Hg⁰), is hardly influenced by other coexistent components, and does not exert an influence on other coexistent components; that is, the reducing catalyst is required to have selectivity for divalent mercury. Specific examples of the reducing catalyst that can be used include zeolite-based catalysts and inorganic compounds such as alkali metal sulfites. Although carbonates and hydroxides can also be used for reducing action, the catalyst is limited to such catalysts due to the coexistence of acidic substances such as SO₂ and NO₂ contained in large amounts in coal combustion exhaust gas. The shape of the reducing catalyst is not particularly limited, but is preferably granular or honeycomb-shaped for less pressure loss and for easy exchange and charging into the reducing catalyst part 3. Not only the catalyst formed in such shape but also the catalyst supported on a carrier having such shape can be used.

The ultraviolet absorption analyzer 10 (not shown) forms an optical system consisting of a ultraviolet light source, a sample cell, a ultraviolet detector and an optical filter, and the quantity of absorption light, in the ultraviolet range, of Hg⁰ in a sample introduced into the sample cell can be detected by the ultraviolet detector to determine the concentration of Hg0 in the sample.

<Another Constitutional Example of the Present Measuring Apparatus>

The present measuring apparatus comprises a sample introducing path for heating and introducing the sample from the present removing apparatus, a reduced-catalyst part charged with a catalyst of an inorganic material having reducing power and being poor in reactivity with acidic substances, a reduced-gas flow path provided with the reducing catalyst part, an oxidizing catalyst part charged with an oxidizing catalyst, an oxidized-gas flow path provided with the oxidizing catalyst part, and an ultraviolet absorption analyzer for measuring mercury concentration by comparing mercury concentration between the reduced gas and oxidized gas. The present measuring apparatus uses the present removing apparatus thereby treating SeO₂ present in exhaust gas, decreasing measurement errors attributable to the interfering influence of coexistent components such as SO₂, NO₂ and moisture in the exhaust gas, and simultaneously securing high selectivity and measurement accuracy in measurement of mercury in coal exhaust gas.

FIG. 11 illustrates another constitution of the present measuring apparatus. This measuring apparatus for measuring total mercury in a sample is constituted such that a scrubber 7 is used as the present removing apparatus (dry treatment) and a differential analyzer is used as an ultraviolet absorption analyzer 10. A reduced gas wherein total mercury is converted into Hg⁰ by selectively reducing mercury in a sample, and an oxidized gas wherein total mercury is converted into Hg²⁺ by selectively oxidizing mercury in the sample, are prepared, and

(a) when there is a single ultraviolet absorption cell (sample cell) in an ultraviolet absorption analyzer, the reduced gas and oxidized gas are introduced alternately into the sample cell, and both of them are compared with respect to the quantity of absorption light, or
(b) when there are plural (usually two) sample cells, the reduced gas and oxidized gas are introduced simultaneously into the sample cells respectively, and both of them are measured for their difference in the quantity of absorption light,
thereby enabling measurement without undergoing the influence of other coexistent components not changed by both oxidation treatment and reduction treatment. Accordingly, one sample is subjected to oxidation and reduction in series or in parallel, and two samples thus obtained by oxidation and reduction respectively are measured for their difference in the state of mercury therein, whereby measurement accuracy can be secured without undergoing the influence of other coexistent gas components.

A sample is collected from a sample inlet 11 by suction with a suction pump 15 arranged downstream of the ultraviolet absorption analyzer 10. The collected sample is cleaned with a dust filter 12, then SeO₂ in the sample is removed with a scrubber 7, and mercury in the sample is selectively reduced in a reducing catalyst part 13, thereby preparing a reduced gas wherein total mercury is converted into Hg⁰. Thereafter, the gas is divided into two via a secondary cooling unit 3 (gas/liquid separator), and one half (flow path a) is passed through a refining tool 16 to remove Hg⁰ in the sample, or mercury in the sample is selectively oxidized, to give an oxidized gas wherein total mercury was converted into Hg²⁺, which is then introduced via valve 17 into an ultraviolet absorption analyzer 10. Other half (flow path b), without being treated, is introduced via valve 17 into the ultraviolet absorption analyzer 10. As the material contacting with the gas, it is possible to use not only inexpensive glass, quartz, and ceramics but also metals such as Ti and anodized SUS.

In usual measurement, the flow paths a and b are switched periodically with valve 17, and from a different between the two, Hg²⁺ is detected with the ultraviolet absorption analyzer 10. At the time of calibration, a zero gas and a span gas are introduced through a calibration gas inlet 18, passes via a flow path d, and introduced into the ultraviolet absorption analyzer 10. As the span gas, a gas containing mercury at a predetermined concentration which is generated in a generator into which a zero gas was introduced (not shown) is used. The valve 17 is switched periodically usually at about 0.5- to 30-second intervals.

The temperature in the sample flow path extending from the sample collection part 11 to the ultraviolet absorption analyzer 10, as shown in Table 2 below, is preset for preventing generation of condensed water in a dust filter 12 etc. and formation of amalgam of mercury with SeO₂ and for keeping the scrubber 7 at a suitable temperature of 150 to 250° C.

TABLE 2

| Pretreatment unit etc. for mercury | | |
|---|---|---|
| measurement | Preset temperature (° C.) | |
| Stack gas | 200~350° | C. |
| Probe tube | 190~200° | C. |
| Dust filter | 190~200° | C. |
| SeO₂ scrubber | 150~250° | C. |
| Hg reducing catalyst | 250~500° | C. |
| Gas/liquid separator | 5~30° | C. |
| Measurement cell | Thermostat bath at 55° | C. |

A refining tool 16 can selectively adsorb and remove Hg⁰ in a sample by using an adsorbent such as activated carbon. For example, a Pt-silica- or Pd-alumina-based catalyst, or a catalyst such as V₂O₅, can be used to oxidize Hg⁰ in a sample into Hg²⁺ not detectable with the ultraviolet analyzer 10, whereby Hg⁰ can be selectively removed. At this time, when an oxidizing catalyst is used as the refining tool 16, the operation temperature can be in the same middle temperature range (for example 300 to 400° C.) as in the reducing catalyst part 3 so that both of them can be housed in the same unit to unify the temperature control system and to downsize the apparatus.

Because a predetermined concentration of Hg gas for calibration or checking cannot be prepared as high-pressure gas, a generator should be used. For example, a predetermined concentration of Hg gas can be obtained by a method of passing a zero gas through a surface layer of Hg kept at a predetermined temperature, or by mixing a zero gas with Hg permeating into a permeation tube dipped in a Hg liquid bath. The predetermined concentration of Hg gas can be diluted with a zero gas to give a low concentration of Hg gas. For feeding a calibration gas, it can be fed through a calibration gas inlet 18 shown in FIG. 11.

Although the ultraviolet absorption analyzer 10 can use the same constitution as in FIG. 10, a constitution forming an optical system consisting of 2 sample cells can be additionally used. When there is a single ultraviolet absorption cell in the analyzer, the reduced gas and oxidized gas are introduced alternately into the sample cell in the ultraviolet absorption analyzer 10, as shown in FIG. 11, and both of them are compared with respect to the quantity of absorption light. On the other hand, when there are two sample cells, the reduced gas and oxidized gas are introduced simultaneously into the sample cells respectively, and both of them are measured for their difference in the quantity of absorption light. This system is used for directly measuring the difference between the two samples because a difference in quantity of absorption between the two can be detected.

According to the constitution described above, the present measuring apparatus can achieve the following technical effects:

(1) In measurement of total mercury in coal combustion exhaust gas, highly sensitive measurement which is accurate and stable for a long time with less interfering influence of coexisting gas SeO₂ can be realized.
(2) SeO₂ that is an interfering component in mercury measurement can be selectively removed.
(3) By arranging a scrubber at a former stage of a reducing catalyst part, there is brought about a protective effect for maintaining the performance of the mercury catalyst. The interfering influence of SeO₂ on the reducing catalyst in a latter stage is prevented.
(4) A scrubber is arranged in a former stage of a reducing catalyst part and the operation temperature is kept at 150 to 250° C. thereby functioning as preheat for the reducing catalyst part, thus enabling effective utilization of the heat.
(5) The operation temperature of the scrubber can be kept in the same degree as the heating temperature of the pretreatment apparatus in mercury measurement, and thus the structure of the apparatus can be simplified.

Other Constitutional Examples of the Present Measuring Apparatus>

Other constitutional examples of the present measuring apparatus can include various constitutions in combination with the present removing apparatus. For example, in the combination with the present removing apparatus shown in FIG. 8, a sample inlet 1, a dust filter 12 and a reducing catalyst part 13 are arranged upstream of a heating conduit 1, and a filter 14, an ultraviolet absorption analyzer 10 and a suction pump 15 are arranged just after scrubber 7, whereby the present measuring apparatus capable of performing the wet treatment and dry treatment in series can be constituted. High efficiency of removal of SeO₂ can be secured for a long time.

As described above, the method and apparatus for measuring mercury according to the present invention have been described mainly by reference to those for measuring mercury in coal combustion exhaust gas, but the present method and apparatus for measuring mercury can also be applied to samples having the same composition as in process gas etc. and for study of various processes. The present invention is particularly useful for measurement of samples containing coexistent SO₂ and metal oxides.

What is claimed is:

1. An apparatus for removing selenium oxide in a sample, comprising:
(1) a heating introduction path for heating a sample;
(2) a primary cooling unit connected to the heating introduction path and having a flow path through which the heated sample flows countercurrently to cooling water, whereby the heated sample is mixed with, and cooled by, cooling water;
(3) a secondary cooling unit connected to the primary cooling unit and having a spiral flow path for cooling the mixed gas and water and having a space for gas/liquid separation at the end of the spiral flow path;
(4) a regenerator connected to the secondary cooling unit for introducing condensed water from the secondary cooling unit and removing SeO₂; and
(5) a condensed water-cooling path for connecting the regenerator to the primary cooling unit.

2. The apparatus for removing selenium oxide in the sample according to claim 1, wherein the primary cooling unit and the secondary cooling unit are provided in a cooling treatment unit having (a) a water feed opening for the cooling water upstream of the spiral flow path, (b) a feed opening of the sample downstream of the water feed opening, (c) a space for gas/liquid separation, arranged at the end of the spiral flow path, (d) a condensed-water discharge flow path and a treated-gas feed flow path, branched from the space, and (e) a cooling means for cooling each of the path flows and the space.

3. The apparatus for removing selenium oxide in the sample according to claim 1, wherein a combination of the primary cooling unit and the secondary cooling unit, and a scrubber are arranged in one of a series and a parallel flow arrangement.

4. The apparatus for removing selenium oxide in the sample according to claim 1, wherein a combination of the primary cooling unit and the secondary cooling unit, or the cooling treatment unit, and the scrubber are arranged in series or in parallel.

5. An apparatus for measuring mercury in coal combustion exhaust gas by using the apparatus for removing selenium oxide in a sample according to claim 1, which comprises a sampling part for collecting a coal combustion exhaust gas as the sample, s sample introducing path for heating and introducing the sample from the sampling part to the removing apparatus, and a mercury analyzer for measuring the amount of mercury.

6. An apparatus for measuring mercury in coal combustion exhaust gas by using the apparatus for removing selenium oxide in a sample according to claim 2, which comprises a sampling part for collecting a coal combustion exhaust gas as the sample, a sample introducing path for heating and introducing the sample from the sampling part, the removing apparatus, and a mercury analyzer.

7. The apparatus for measuring mercury in coal combustion exhaust gas according to claim 6, comprising a reduction catalyst part charged with a catalyst of an inorganic material having reducing effect on mercury and slight reactivity with acidic substances, a reduced-gas flow path provided with the reduction catalyst part, an oxidation catalyst part charged with an oxidation catalyst, an oxidized-gas flow path provided with the oxidation catalyst part, and an ultraviolet absorption analyzer for measuring mercury concentration by comparison between the reduced gas and the oxidized gas.

8. An apparatus for removing selenium oxide in a sample, which comprises:
an introduction path for heating the sample;
a scrubber charged with one of a barium compound, an iron oxide, or a mixture of the barium compound and the iron oxide; and
a heating means for keeping the scrubber at a predetermined temperature, in order to selectively remove selenium oxide.

9. An apparatus for measuring mercury in coal combustion exhaust gas by using the apparatus for removing selenium oxide in a sample according to claim 8, which further comprises a sampling part for collecting a coal combustion exhaust gas as the sample, a sample introducing path for heating and introducing the sample from the sampling part, the removing apparatus, and a mercury analyzer.

10. The apparatus for measuring mercury in coal combustion exhaust gas according to claim 9, comprising:
a reduction catalyst part charged with a catalyst of an inorganic material having reducing power toward mercury and being poor in reactivity with acidic substances, a reduced-gas flow path provided with the reduction catalyst part, an oxidation catalyst part charged with an oxidation catalyst, an oxidized-gas flow path provided with the oxidation catalyst part, and an ultraviolet absorption analyzer for measuring mercury concentration by comparison between the reduced gas and the oxidized gas.

11. A method of removing selenium oxide in a sample, comprising:
heating the sample;
providing a primary cooling treatment wherein the heated sample is cooled by mixing the sample with a cooling water;
providing a secondary cooling treatment wherein the mixed sample is subjected to a gas/liquid separation and simultaneously further cooled to remove the selenium oxide from the sample;
regenerating the cooling water recovered by the secondary cooling treatment to remove selenium oxide;
and reutilizing the regenerated cooling water by cycling as cooling water to the primary cooling treatment.

12. The method of removing selenium oxide in the sample according to claim 11, wherein a combination of the primary cooling treatment and the secondary cooling treatment, for the treatment for selective removal of selenium oxide, are carried out in series or in parallel.

13. A method of measuring mercury in coal combustion exhaust gas by using the method for removing selenium oxide in a sample according to claim 12, which comprises treating, by the removing method, a coal combustion exhaust gas as a measurement sample collected by a sampling part and measuring it with a mercury analyzer.

14. The method of measuring mercury in coal combustion exhaust gas according to claim 13, wherein the sample is measured with an ultraviolet absorption analyzer and compares with a reduced gas, and wherein mercury in the sample is further reduced with a catalyst consisting of an inorganic material having reducing power, and an oxidized gas, and the sample gas is oxidized with an oxidation catalyst.

15. The method of removing selenium oxide in the sample according to claim 11 wherein the sample is heated to a temperature within a range of 100° C. to 200° C.

16. The method of removing selenium oxide in the sample according to claim 15 wherein the heated sample is cooled to a temperature within a range of 0° C. to 30° C.

17. The method of removing selenium oxide in the sample in claim 16 wherein a sufficient amount of water is counter flowed in the primary cooling treatment to dissolve the SeO₂ in the water and to suppress the formation of H₂SeO₃.

18. The method of removing selenium oxide in the sample in claim 17 wherein the secondary cooling treatment separates a gas in the samples from the SeO₂ by cooling a spiral flow path to prevent droplet formation and the removal of the water with dissolved SeO₂.

19. The method of removing selenium oxide in the sample of claim 11 wherein the primary cooling treatment and the secondary cooling treatment are simultaneously conducted when an amount of the sample is relatively small.

20. A method of monitoring mercury in coal combustion exhaust gas comprising the steps of:
providing a sample of coal combustion exhaust gas with mercury and SeO₂;
heating the sample to a temperature that retards condensation of SeO₂ from the sample;
mixing the heated sample with cooling water to dissolve the SeO₂ in the water;
separating a portion of the sample with mercury as a gas from the water with the dissolved SeO₂; and
measuring the portion of the sample with mercury with a mercury analyzer to determine the amount of mercury in the coal combustion exhaust gas.

21. The method of claim 20 wherein the sample is heated to a temperature within a range of 100° C. to 200° C.

22. The method of claim 20 wherein the mixing step of cooling water cools the sample to ambient temperature.

23. The method of claim 20 wherein the separating step keeps the portion of the sample with mercury at a temperature that prevents dew formation.

24. The method of claim 20 wherein the mixing step and the separating step can be performed in a common cooling treatment unit as a single step.

25. The method of claim 20 wherein the water with the dissolved SeO₂ is subject to a regeneration step for removing selenious acid with anion-exchange resin and selenious-acid adsorbents.

26. The method of claim 20 wherein the measuring step is performed with an ultraviolet absorption analyzer by comparison with a reduced gas, wherein mercury in the sample is reduced with a catalyst consisting of an inorganic material having reducing power, and an oxidized gas, wherein the sample gas is oxidized with an oxidation catalyst.
